(19) United States
(12) Patent Application Publication
(10) Pub. No.: US 2021/0121237 A1
(43) Pub. Date: Apr. 29, 2021

(54) SYSTEMS AND METHODS FOR AUGMENTED REALITY DISPLAY IN NAVIGATED SURGERIES

(71) Applicant: INTELLIJOINT SURGICAL INC., KITCHENER (CA)

(72) Inventors: RICHARD TYLER FANSON, STONEY CREEK (CA); ANDRE NOVOMIR HLADIO, WATERLOO (CA); RAN SCHWARZKOPF, NEW ROCHELLE, NY (US); JONATHAN SMITH, KITCHENER (CA); LUKE ADRIAN WEBER BECKER, KITCHENER (CA)

(21) Appl. No.: 16/494,540

(22) PCT Filed: Mar. 16, 2018
(86) PCT No.: PCT/CA2018/050323

§ 371 Date: Sep. 16, 2019

Related U.S. Application Data
(60) Provisional application No. 62/472,705, filed on Mar. 17, 2017.

(51) Int. Cl. A61B 34/20

(52) U.S. Cl. CPC A61B 34/20; A61B 2090/365

(57) ABSTRACT

Systems and methods describe augmented reality provided for navigated surgery. An augmented reality overlay (e.g. computer generated images) is rendered and displayed over images of a tracked anatomical structure. An optical sensor unit provides tracking images of targets associated with objects including the anatomical structure in a real 3D space as well as visible images thereof. The anatomical structure is registered, generating corresponding poses of the anatomical structure in a computational 3D space from poses in the real 3D space. The overlay pose in the computational 3D space is aligned with the anatomical structure pose so that the overlay is rendered on a display of the anatomical structure in a desired pose. The overlay may be generated from a (3D) overlay model such of a generic or patient specific bone, or other anatomical structure or object. The overlay may be used to register the anatomical structure.

### CROSS REFERENCE

This application claims the domestic benefit within the United States of, and Paris Convention priority otherwise to, U.S. Provisional Patent Application No. 62/472,705, filed Mar. 17, 2017, the entire contents of which are incorporated herein by reference where permitted.

### FIELD

This disclosure relates to navigate surgeries where the poses of objects such as surgical tools, prosthetics and portions of patient anatomy (e.g. bones) are tracked and information is determined and displayed to assist with a procedure and more particularly to systems and methods for augmenting reality such as by overlaying computer generated images on real time visible images of the procedure.

### BACKGROUND

Navigational surgery systems using various modalities such as optical, electromagnetic, etc. are used in surgical procedures to obtain information about spatial localization of objects (e.g. rigid bodies and the patient's anatomy). Information may be displayed on a display screen in real time during a surgical procedure to assist the surgeon or other professional.

Navigational surgery systems perform a registration of the object(s) being tracked in a real 3D space to a co-ordinate frame (e.g. a computational 3D space) maintained by the system. In this way the pose (position and orientation) of the objects may be computationally known and may be related to one another in the system. Relative pose information may be used to determine various measurements or other parameters about the objects in the real 3D space.

### SUMMARY

Systems and methods are provided for augmenting the reality of a navigated surgery in relation to a patient. An augmented reality (AR) overlay (e.g. computer generated images) is rendered and displayed over images of the patient as an anatomical structure is tracked. An optical sensor unit provides the system with tracking images of targets associated with objects in its field of view of the procedure in a real 3D space as well as visible images thereof. The system registers the anatomical structure, generating corresponding poses of the anatomical structure in a computational 3D space from poses in the real 3D space. The pose of the overlay in the computational 3D space is aligned with the pose of the anatomical structure so that when rendered and provided to a display of the anatomical structure the overlay is in a desired position. The overlay may be generated from an overlay model such as a 3D model of an object or a generic or patient specific bone or other anatomical structure. The augmented reality overlay may be useful to assist with registration of the anatomical structure, for example, by moving a tracked anatomical structure into alignment with the overlay as rendered on a display or by maintaining a position of the anatomical structure and moving the overlay by moving a tracker in the real 3D space that is associated to the overlay in the computational 3D space. Once aligned a lock operation captures a pose and registers the anatomical structure. Thereafter the overlay is aligned to the pose of the structure as it is tracked.

There is provided a computer-implemented method to provide augmented reality in relation to a patient, the method comprising: receiving, by at least one processor, images of a real 3D space containing the patient and one or more targets associated with respective objects and/or anatomical structures of the patient in the real 3D space, the images received from a single optical sensor unit having a field of view of the real 3D space containing the patient and one or more targets; determining tracking information from the images for respective ones of the one or more targets; registering an anatomical structure of the patient in a computational 3D space maintained by the at least one processor using tracking information for a respective target associated with the anatomical structure, generating a corresponding position and orientation of the anatomical structure in the computational 3D space from a position and orientation of the anatomical structure in the real 3D space; aligning an overlay model of an augmented reality overlay to a desired position and orientation in the computational 3D space relative to the corresponding position and orientation of the anatomical structure; and rendering and providing the augmented reality overlay for display on a display screen in the desired position and orientation.

The method may comprise providing the images of the real 3D space for display on the display screen to simultaneously visualize the anatomical structure and the augmented reality overlay.

The optical sensor unit may comprises calibration data to determine 3D measurements from the images of the real 3D space provided by the optical sensor unit in 2D and the step of determining tracking information comprises using by the at least one processor the calibration data to determine the tracking information.

The method may comprise, in real time and in response to a relative movement of the anatomical structure and the optical sensor unit in the real 3D space, wherein a pose of the respective target associated with the anatomical structure continuously indicates a position and orientation of the anatomical structure in the real 3D space: determining a moved position and orientation of the anatomical structure in the real 3D space using the images received from the optical sensor unit; updating the aligning of the augmented reality overlay relative to the moved position and orientation of the anatomical structure to determine a moved desired position and orientation of the augmented reality overlay; and providing the augmented reality overlay for display in the moved desired position and orientation. The respective target associated with the anatomical structure is either 1) attached to the anatomical structure such that one or both of the optical sensor unit and anatomical structure are free to move in the real 3D space or 2) attached to another object while the location of anatomical structure remains constant in the real 3D space and the optical sensor unit alone is free to move in the real 3D space.

The image of the real 3D space may comprise an enlarged image and the augmented reality overlay enlarged to match the enlarged image.

The anatomical structure may be a femur and one of the targets associated with the anatomical structure is a femoral target attached to the femur. The overlay model may be a 3D model of a generic or a patient-specific femur model and the augmented reality overlay is an image representing a generic or a patient-specific femur respectively.

The anatomical structure is a pelvis and one of the targets associated with the anatomical structure is a pelvic target. The overlay model may be a 3D model of a generic or a patient-specific pelvis model and the augmented reality overlay is an image representing a generic or a patient-specific pelvis respectively.

The overlay model may be a mechanical axis model and the augmented reality overlay is an image of a mechanical axis and/or a further axis or plane, a location of which is determined relative to a location of the mechanical axis of the anatomical structure. The method may comprise determining the mechanical axis of the anatomical structure using tracking information obtained from target images as the anatomical structure is rotated about an end of the anatomical structure. The further axis and/or plane may be a resection plane. The location of the resection plane along the mechanical axis model may be adjustable in response to user input thereby to adjust the desired position and orientation of the resection plane in the augmented reality overlay. The bone may be a femur. The method may comprise: registering a tibia of a same leg of the patient in the computational 3D space, the tibia coupled to a tibia target of the one or more targets, the at least one processor determining a position and orientation of the tibia in the real 3D space to generate a corresponding position and orientation of the tibia in the computational 3D space from tracking information determined from images of the tibia target; aligning a second overlay model of a second augmented reality overlay to a second desired position and orientation in the computational 3D space relative to the corresponding position and orientation of the tibia; providing the second augmented reality overlay for display on a display screen in the second desired position and orientation. Registering the tibia may use images of one of the targets attached to a probe where the probe identifies first representative locations on the tibia with which to define a first end of the tibia and second identifying locations about an ankle of the patient with which to define a second end and a mechanical axis of the tibia. The method may comprise: tracking movement of the position and orientation of the tibia in the real 3D space; updating the corresponding position and orientation of the tibia in response to the movement of the position and orientation of the tibia in the real 3D space; updating the aligning of the second augmented reality overlay relative to the position and orientation of the tibia as moved to determine the second desired position and orientation as moved; and providing the second augmented overlay for display in the second desired position and orientation as moved. The method may comprise determining a location of each of the augmented reality overlay of the femur and the augmented reality overlay of the tibia and indicating a relative location to one another to denote at least one of proximity and intersection.

The optical sensor unit may be configured in accordance with one of the following: a) multi-spectral camera (providing visible and tracking channels); (b) dual cameras (providing respective visible and tracking channels); (c) dual imager (using prism to split visible and tracking channels); and (d) tracking channel using visible light.

The anatomical structure may be surgically modified and the overlay model may be a 3D model of a generic or patient-specific human anatomical structure prior to replacement by the prosthetic implant and the augmented reality overlay is an image representing a generic or a patient-specific human anatomical structure respectively. The method may comprise providing images of the patient for display on the display screen to simultaneously visualize the anatomical structure and the augmented reality overlay.

The overlay model may be a patient-specific model defined from pre-operative images of the patient.

Images of the patient may show a diseased human anatomical structure and the overlay model may represent the diseased human anatomical structure without a disease.

There is provided a computer-implemented method to provide augmented reality in relation to a patient, the method comprising: receiving, by at least one processor, images of a real 3D space containing the patient and one or more targets associated with respective objects and/or anatomical structures of the patient in the real 3D space, the images received from a single optical sensor unit having a field of view of the real 3D space containing the patient and one or more targets; determining tracking information from the images for respective ones of the one or more targets; providing, for simultaneous display on a display screen, i) images of the real 3D space from the optical sensor; and ii) renderings of an augmented reality overlay; wherein the augmented reality overlay is defined from an overlay model and displayed in an initial position and orientation within the field of view of the optical sensor unit as displayed on the display screen; registering, by the at least one processor, an anatomical structure of the patient in the computational 3D space by receiving input to use tracking information to capture a pose of one of the targets in the field of view, the one of the targets attached to the anatomical structure, the input received when the anatomical structure as displayed is aligned with the initial position and orientation of the augmented reality overlay; and wherein the pose defines a position and orientation of the anatomical structure in the real 3D space to generate a corresponding position and orientation of the anatomical structure in the computational 3D space; and associating, in the computational 3D space, a desired position and orientation of the augmented reality overlay relative to the corresponding position and orientation of the anatomical structure.

There is provided a computer-implemented method to provide augmented reality in relation to a patient, the method comprising: receiving, by at least one processor, images of a real 3D space containing the patient and one or more targets associated with respective objects and/or anatomical structures of the patient in the real 3D space, the images received from a (single) optical sensor unit having a field of view of the real 3D space containing the patient and one or more targets; determining tracking information from the images for respective ones of the one or more targets; providing, for simultaneous display on a display screen, i) optical sensor images of the real 3D space from the optical sensor; and ii) renderings of an augmented reality overlay; wherein the augmented reality overlay is defined from an overlay model and displayed in an overlay position and orientation relative to a pose of an overlay target in the field of view of the optical sensor, the overlay position and orientation moving in response to movement of the overlay target in the real 3D space; registering, by the at least one processor, an anatomical structure of the patient in the computational 3D space by receiving input to use tracking information to capture a registration lock pose of the overlay target and a registration pose of an anatomical structure target associated with the anatomical structure, the input received when said augmented reality overlay is aligned with an initial position and orientation of the anatomical structure in the real 3D space, generating a corresponding position and orientation of the anatomical structure in the computational 3D space from the initial position and orientation of the anatomical structure in the real 3D space; and associating, in the computational 3D space, a desired position and orientation of the augmented reality overlay relative to the corresponding position and orientation of the anatomical structure for use when subsequently rendering the augmented reality overlay.

In association with these methods for registering using the overlay, the methods may respectively further comprise, in real time and in response to a relative movement of the anatomical structure and the optical sensor unit in the real 3D space, wherein a pose of the anatomical structure target associated with the anatomical structure continuously indicates a position and orientation of the anatomical structure in the real 3D space: determining a moved position and orientation of the anatomical structure using the images received from the optical sensor; updating the aligning of the augmented reality overlay relative to the moved position and orientation of the anatomical structure to determine a moved desired position and orientation of the augmented reality overlay; and rendering and providing, for simultaneous display on the display screen, i) images of the real 3D space from the optical sensor; and ii) the augmented reality overlay in response to the moved desired position and orientation of the augmented reality overlay.

The methods may respectively further comprise performing an initial registration of the anatomical structure, an initial aligning of the augmented reality overlay to the anatomical structure and an initial rendering and providing such that the augmented reality overlay and anatomical structure are misaligned in the images of the 3D space when displayed.

There is provided a computer-implemented method to provide augmented reality in relation to a patient where the method comprises receiving, by at least one processor, images of a real 3D space containing the patient, a bone removal tool and a target associated with an anatomical structure of the patient in the real 3D space, the images received from a single optical sensor unit having a field of view of the real 3D space containing the patient and the target; determining tracking information from the images for the target; registering the anatomical structure of the patient in a computational 3D space maintained by the at least one processor using tracking information for a respective target associated with the anatomical structure, generating a corresponding position and orientation of the anatomical structure in the computational 3D space from a position and orientation of the anatomical structure in the real 3D space; aligning an overlay model of an augmented reality overlay comprising a planned implant position to a desired position and orientation in the computational 3D space relative to the corresponding position and orientation of the anatomical structure; and rendering and providing the planned implant position and the images of the real 3D space for display on a display screen to simultaneously visualize the planned implant position and the bone removal tool.

There is provided a computer-implemented method to provide augmented reality in relation to a patient, where the method comprises: receiving, by at least one processor, images of a real 3D space containing the patient and one or more targets associated with respective objects and/or anatomical structures of the patient in the real 3D space, the images received from a single optical sensor unit having a field of view of the real 3D space containing the patient and one or more targets; determining tracking information from the images for respective ones of the one or more targets; registering an anatomical structure of the patient in a computational 3D space maintained by the at least one processor using tracking information for a respective target associated with the anatomical structure, generating a corresponding position and orientation of the anatomical structure in the computational 3D space from a position and orientation of the anatomical structure in the real 3D space; registering one or more of: a surgical plan and a tool; aligning respective overlay models of augmented reality overlays to desired positions and orientations in the computational 3D space relative to the corresponding positions and orientations of the anatomical structure, the surgical plan and/or the tool; determining desired display information based on receiving user input or context information; and selectively, based on desired display information, rendering and providing the augmented reality overlays for display on a display screen in the desired positions and orientations.

There is provided a navigational surgery system comprising a computing unit, an optical sensor unit and one or more targets for tracking objects by the optical sensor unit providing tracking images having tracking information for said targets and visible images of a procedure in a field of view of the optical sensor unit to the computing unit, the computing unit having at least one processor configured to perform a method in accordance with any one of the methods herein. The navigational surgery system may include a platform to selectively, removably and rigidly attach one of the optical sensor unit and one of the trackers to an anatomical structure of the anatomy of the patient, the platform comprising a body having at least one surface, the at least one surface configured to provide an optically trackable pattern, a repeatable optical sensor mount and a repeatable target mount, wherein the optically trackable pattern extends into a field of view of the optical sensor unit when mounted to the platform. The spatial relationship between the optically trackable pattern and the repeatable target mount is predefined by a target-pattern definition. The computing unit may be configured to: receive first images including the optically trackable pattern features when the optical sensor unit is mounted to the platform; perform operations to calculate a pose of the optically trackable pattern; perform operations to calculate the pose of the repeatable target mount based on the pose of the optically trackable pattern and the target-pattern definition; receive second images when the optical sensor unit is removed from the platform and one of the trackers is mounted to the platform, the second images including the one of the trackers mounted to the platform; and track the anatomical structure to which the one of the trackers is attached.

It will be understood that also provided are platform aspects as well as computer program product aspects where a device stores instructions in a non-transitory manner to configure a system, when the instructions are executed by at least one processor thereof, to perform any of the methods.

Reference in the specification to “one embodiment”, “preferred embodiment”, “an embodiment”, or “embodiments” (or “example” or “examples”) means that a particular feature, structure, characteristic, or function described in connection with the embodiment/example is included in at least one embodiment/example, and may be in more than one embodiment/example if so capable. Also, such phrases in various places in the specification are not necessarily all referring to the same embodiment/example or embodiments/examples.

### DESCRIPTION OF THE DRAWINGS

FIG. 1 is a representation of a navigational surgery system.

FIG. 2 is a representation of an axis frame for registration in navigational surgery system of FIG. 1.

FIG. 3 is a flowchart of a method of registration according to one example.

FIG. 4 is a screenshot showing a pelvic overlay in a mock surgery.

FIG. 5 illustrates a flowchart of operations for providing augmented reality relative to a patient according to an example.

FIG. 6A is a screenshot of a GUI showing a captured video image displayed with an overlay and FIG. 6B is a sketch of the video image and overlay of FIG. 6A where stippling is enlarged for clarity.

FIG. 7 is a captured video image, for display in a GUI such as shown in FIG. 6A, with a cutting plane overlayed as guidance in a mock total knee arthroplasty.

FIGS. 8A and 8B are respective captured video images, for display in a GUI such as shown in FIG. 6A, showing a target coupled to knee anatomy (e.g. a femur) as a knee moves from extension to flexion showing mechanical axis and resection plane over the real time images of the knee.

FIGS. 9A and 9B: are screenshots showing use of a probe to trace anatomy in 3D space and leave markings which could be used as an AR overlay.

FIG. 10 illustrates a flowchart of operations to provide augmented reality in relation to a patient in accordance with one example to achieve registration.

FIG. 11 illustrates a flowchart of operations to provide augmented reality in relation to a patient in accordance with one example to achieve registration.

FIG. 12A shows a sketch of an operating room including a camera (e.g. an optical sensor unit) tracking an anatomical structure via a tracker and a surgical tool in accordance with an example.

FIG. 12B is an illustration of a display screen 1220 showing a video image of the operating room of FIG. 12A including an overlay in accordance with an example.

FIG. 13A is a top perspective view of an AR platform in accordance with an example.

FIGS. 13B-C are side views of the AR platform showing how to use the AR platform of FIG. 13A to facilitate optical sensor unit attachment to an anatomical structure in accordance with an example.

### DETAILED DESCRIPTION

A navigational surgery system provides spatial localization of a rigid body (such as, instruments, prosthetic implants, anatomical structures etc.) with respect to another rigid body (such as, another instrument, a patient's anatomy etc.). Examples of navigational surgery systems and associated methods are described in greater detail in PCT/CA2014/000241 titled “System and Method for Intra-operative Leg Position Measurement” by Hladio et al filed Mar. 14, 2014, the entire contents of which are incorporated herein by reference. Navigational surgery systems may have various modalities including optical technology and may use active or passive targets to provide pose (position and orientation) data of the rigid body being tracked. As noted herein below, an optical based system providing images which include tracking information and visible images of the procedure may be augmented with overlays to assist with the procedure. Visible images are those which primarily comprise images from the visible light spectrum and which may be displayed on a display for perception by a human user.

Various methods to register objects, particularly patient anatomy are known. US Pat. Appln. Publication No. US20160249987A1 published 2016 Sep. 1 and entitled “Systems, methods and devices for anatomical registration and surgical localization” incorporated herein by reference describes some registration methods. As noted therein, it is desirable that a method of registration be fast, so as to not undesirably increase the duration of the surgical workflow, and be sufficiently accurate.

Described herein below are additional registration methods using augmented reality to assist with this step to enable tracking operations.

### Augmented Reality in Navigational Systems

An augmented reality overlay (e.g. comprising a computer generated image) on a real time visible image of a surgical procedure may be presented via a display to a surgeon or other user to provide an augmented reality view of a surgical procedure. Though described with reference to a navigational surgery system, it is understood that such systems may be useful in clinic or other settings and need not be used exclusively for surgery but may also be used for diagnostic or other treatment purposes.

The augmented reality overlay may be generated from a 3D model of an object to be displayed or form other shape and/or positional information. The object may be defined from medical image data, which may be segmented or pre-processed. The medical image data may represent generic or patient specific anatomy such as a bone or other anatomical structure. The overlay model may be constructed from 3D images of the anatomy. Patient specific images may be generated from CT, MRI or other scanning modalities, etc. Generic overlay models may be constructed from scans of anatomy (e.g. of other patients or bodies) or from CAD or other computer models and/or renderings, etc.

The anatomy represented in an overlay may be diseased anatomy and such may be displayed over the patient's actual anatomy or a prosthesis. The anatomy represented may be healthy or pre-diseased anatomy constructed from the patient's diseased anatomy as described below.

Other objects for display may be surgical tools (e.g. jigs), or representations of shapes, lines, axis and/or planes (e.g. of patient anatomy or for cutting), or other geometrical features, etc.

Overlays may include target parameters. Target parameters may be based on a surgical plan (i.e. same type of plan surgeons do today). A benefit is that such parameters allow a practitioner to visualize the plan better, with reference to the actual patient (not just relative to a medical image). Target parameters may be based desired/planned location of an implant. Total Hip Arthroplasty (THA) examples include acetabular cup angle, hip center of rotation, resection plane for femoral head. Knee examples include resection plane for distal femur and/or proximal tibia. Spine examples include location of pedicle screw within vertebral body. Target parameters may include a location of targeted anatomy. Neurosurgical examples include a location of tumour within brain.

Overlays may be generated, e.g. during the procedure, based on tracking data collected by the navigational surgery system and may comprise (a) 3D scans (e.g. structured light such as from a laser may be projected onto the surface of the patient and detected by the optical sensor unit to define a 3D scan)) and (b) 3D “drawings”.

Real time visible images are obtained from an optical sensor unit coupled to a computing unit of the system, which optical sensor unit provides both visible images of the procedure as well as tracking information (tracking images) for tracking objects in a field of view of the optical sensor. Optical sensors often use infrared based sensing technology for sensing targets coupled to objects being tracked. To provide both tracking images (i.e. tracking information) and visible images the optical sensor unit may be configured in accordance with one of the following:

multi-spectral camera (providing visible and tracking channels)

dual cameras (e.g. providing respective visible and tracking channels)

dual imager (using prism to split visible and tracking channels)

tracking channel uses visible light

The optical sensor unit may be configured as a single unit. When capturing separate tracking images and visible images, it is preferred that the field of view of a camera or imager capturing tracking images be the same as the field of view a camera or imager capturing the visible images so as not to require alignment of the tracking images and visible images.

In some embodiments, the augmented reality overlay is displayed in association with an anatomical structure of the patient that is tracked by the tracking system. As the relative pose of the anatomical structure moves with respect to the optical sensor unit (e.g. because the structure moves or the optical sensor unit moves) and thus the structure moves within the real time image, the overlay may track with the anatomical structure and similarly move when displayed.

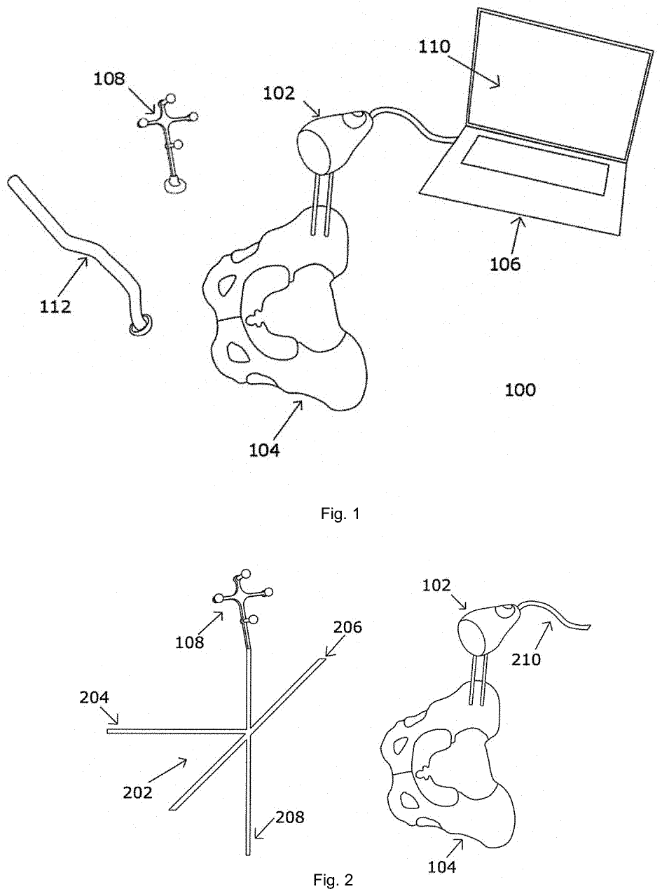

FIG. 1 illustrates a navigational surgery system 100, used in THA, where an optical sensor unit 102 is attached an anatomy of a patient (e.g. a pelvis 104) and communicates with a workstation or an intra-operative computing unit 106. The pose (position and orientation) of a target 108 can be detected by the optical sensor unit 102 and displayed on a graphical user interface (GUI) 110 of the intra-operative computing unit 106. The target 108 may be attached to an instrument 112 or to a part of the anatomy of the patient (e.g. to a femur). In some embodiments, removable targets are used. System 100 may be used in other procedures and may be adapted accordingly, for example, by use of different instruments, attachment of the optical sensor unit to different anatomical structures or other surfaces (e.g. off of the patient).

Within system 100, optical sensor unit 102 provides both real time images from its field of view as well as tracking information for target(s) in the field of view.

In order to provide electronic guidance with respect to the anatomy of the patient in THA, the spatial coordinates of the anatomy of the patient (e.g., the pelvis) with respect to the system 100 are required. Registration is performed to obtain such coordinates. Anatomical registration pertains to generating a digital positional or coordinate mapping between the anatomy of interest and a localization system or a navigational surgery system. Various methods are known and reference may be made to US Pat. Appln. Publication No. US20160249987A1, for example, where an axis frame is utilized. The method therein is repeated briefly herein.

Pelvic registration, particularly useful in THA, is selected as an exemplary embodiment; however, this description is intended to be interpreted as applicable to general anatomy and in various other surgeries. In this disclosure, normally a sensor is attached to a bone of the anatomy of the patient or a steady surface such as an operating table. A target, detectable by the sensor in up to six degrees of freedom, is located on an object being tracked, such as another bone of the anatomy of the patient, a tool, a prosthesis, etc. However, in general, the locations of the sensor and target can be reversed without compromising functionality (e.g. fixing the target on the bone or a steady surface and attaching the sensor to the object to be tracked), and this disclosure should be interpreted accordingly. It will be understood that an optical sensor unit may be mounted on or off of the patient, on a surgeon or other member of the procedure team, for example on a head or body or hand held. An ability to survey the anatomy from different angles (fields of view) may be advantageous. In some embodiments, the optical sensor unit may be on an instrument/tool or a robot. In some embodiments, the optical sensor, computing unit and display may be integrated as a single component such as a tablet computer. In some embodiments, the optical sensor unit and display may be integrated or remain separate but be configured for wearing by a user such as on a head of the user.

Reference is now made to FIG. 2, which illustrates a device, referred to as an axis frame 202 that may be used to register an anatomy of a patient. Through its shape, the axis frame 202 can define axes, such as a first axis 204, a second axis 206 and a third 208 axis. For example, an axis frame may be comprised of three orthogonal bars that define the three axes. Optical sensor unit 102 is attached to the pelvis 104 of the anatomy of the patient and communicates with an intra-operative computing unit 106 through a cable 210. Optical sensor unit tracks positional information of the target 108 attached to the axis frame 202. This information is used to measure the directions of the anatomical axes of a patient in order to construct the registration coordinate frame. At the time of use, the positional relationship between the axes of the axis frame 202 and the target 108 is known to the intra-operative computing unit 106, either through precise manufacturing tolerances, or via a calibration procedure.

When the axis frame is aligned with the patient, the target 108 thereon is positioned within the field of view of the optical sensor unit 102 in order to capture the pose information (from the target). This aspect may take into account patient-to-patient anatomical variations, as well as variations in the positioning of the optical sensor unit 102 on the pelvis 104. Optical sensor unit 102 may comprise other sensors to assist with pose measurement. One example is accelerometers (not shown). In addition or alternative to accelerometers, other sensing components may be integrated to assist in registration and/or pose estimation. Such sensing components include, but are not limited to, gyroscopes, inclinometers, magnetometers, etc. It may be preferable for the sensing components to be in the form of electronic integrated circuits.

Both the axis frame 202 and the accelerometer may be used for registration. The optical and inclination measurements captured by the system 100 rely on the surgeon to either accurately position the patient, or accurately align the axis frame along the axis/axes of an anatomy of a patient, or both. It may be desirable to provide further independent information for use in registering the anatomy of the patient. For example, in THA, the native acetabular plane may be registered by capturing the location of at least three points along the acetabular rim using a probe attached to a trackable target. When positioning implants with respect to the pelvis, information may be presented with respect to both registrations—one captured by the workstation from optical measurements of the axis frame and inclination measurements (primary registration coordinate frame), and the other captured by the workstation using the reference plane generated from the optical measurements of the localized landmarks on the acetabular rim of the patient (secondary registration coordinate frame)—either in combination, or independently.

It will be understood that the location of the optical sensor unit 102 may be located to another location from which it can detect the position and orientation of one or more targets. For example, the optical sensor unit 102 may be attached to an operating table, held in the hand of a surgeon, mounted to a surgeon's head, etc. A first target may be attached to the pelvis of the patient, and a second target may be attached to a registration device (e.g. a probe or axis frame). The optical sensor unit 102 captures the position and orientation of both targets. The workstation calculates a relative measurement of position and orientation between both targets. In addition, the optical sensor unit 102 captures the inclination measurements, and the position and orientation of the first target attached to the anatomy of the patient. The workstation then calculates the direction of the gravity with respect to the first target. Using the relative pose measurement between both targets, and the direction of gravity with respect to the first target attached to the anatomy of the patient, the workstation can construct the registration coordinate frame in up to six degrees of freedom (6DOF).

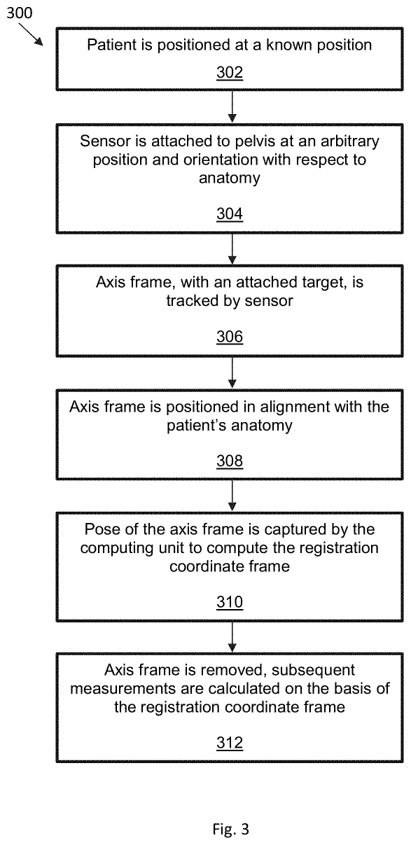

An exemplary method of use, operations 300 of which are shown in the flowchart of FIG. 3, may include the following: at step 302, a patient is positioned, the position being known to the surgeon. At step 304, a sensor is rigidly attached to the pelvis at an arbitrary position and orientation with respect to the anatomy. At step 306, an axis frame, with a trackable target, is tracked by the sensor. At step 308, when the axis frame is positioned in alignment with the known position of the patient's anatomy by the surgeon, step 310 is carried out. The computing unit captures the pose of the axis frame. This pose is used to compute a registration coordinate frame in 6 DOF between the sensor and the anatomy. At step 312, the axis frame is removed and/or discarded, and subsequent positional measurements of the localizer system are calculated on the basis of the registration coordinate frame.

The registration coordinate frame provides a computational 3D space in 6 DOF that is related to the real 3D space in the field of view of the optical sensor unit 102. The registration generates a corresponding position and orientation of the anatomical structure in that computational 3D space from the pose data received from the images of the real 3D space.

Optical sensor unit 102 may provide configuration/calibration data to system 100 for relating the 2D images of the targets received from the sensor to 3D pose information to construct the registration. In some embodiments, the lens or lenses in the optical sensor unit are “fish eye” type lenses. Consequently, a straight line in real 3D space may look non-straight in the images of the real 3D space (due to fish-eye distortion). It may be advantageous to unwarp the image prior to display, based on the calibration data so that straight lines appear straight in the image and curved lines are correctly curved. Alternatively, when rendering an augmented reality overlay, rendering may apply the sensor's distortion model (again, represented by the calibration data) to make straight 3D models appear non-straight according to how the sensor records/captures the real 3D space.

Once registration is achieved, the augmented reality overlay may be aligned to a desired position and orientation in the computational 3D space relative to the anatomical structure's position in the computational 3D space. For an augmented reality overlay that is modeled by a 3D model this may align the overlay model to that space. To align the overlay model may comprise computing a sufficient transformation (e.g. a matrix) to transform the pose of the model data to the desired pose. The augmented reality overlay is then rendered and provided for display on a display screen in the desired position and orientation.

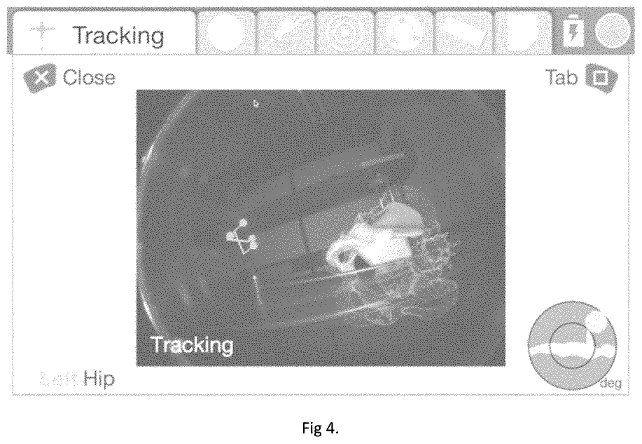

As seen in FIG. 4 where a pelvis overlay is shown, the desired pose of the overlay may be the pose of the anatomical structure, for example, so that the overlay is displayed over the real time image of the anatomical structure in the display.

Other pelvic overlays (not shown) in THA may include target cup position.

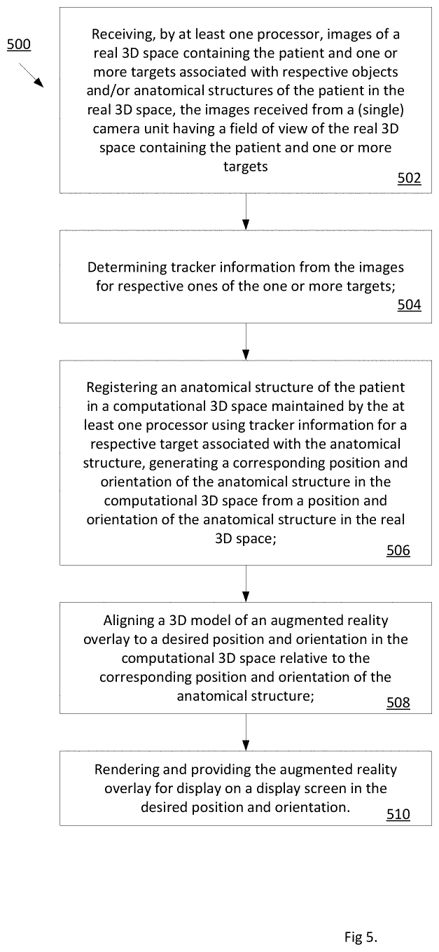

FIG. 5 illustrates a flowchart of operations 500 for providing augmented reality relative to a patient according to an embodiment. At step 502, operations receive, by at least one processor, images of real 3D space containing the patient and one or more targets associated with respective objects and/or anatomical structures of the patient in the real 3D space, the images received from a (single) camera unit have a field of view of the real 3D space containing the patient and one or more targets. At step 504, operations determine tracker information from the images for respective ones of the one or more targets. At step 506, operations register an anatomical structure of the patient in a computational 3D space maintained by the at least one processor using tracker information for a respective target associated with the anatomical structure, generation a corresponding position and orientation of the anatomical structure in the computational 3D space from a position and orientation of the anatomical structure in the real 3D space.

At step 508, operations align a 3D model of an augmented reality overlay to a desired position and orientation in the computation 3D space relative to the corresponding position and orientation of the anatomical structure. At step 510, operations render and provide the augmented reality overlay for display on a display screen in the desired position and orientation.

The display of the overlay may be useful to verify that registration is correct. If the overlay is not aligned in the display as expected, registration may be repeated in a same or other manner. Different types of overlays may be aligned in respective manners. For example, bone based overlays align with a respective patient bone. A plane or axis based overly aligns with a patient plane or axis, etc. As further described below, an augmented reality overlay may be used to perform registration in accordance with further methods.

It will be appreciated that once registered, the relative pose of the optical sensor unit and anatomical structure may change. For example, if a target is attached to the pelvis or otherwise associated thereto (i.e. there is no relative movement between target and object being tracked), the optical sensor unit may move to change its field of view. Provided that the target remains in the field of view, the pelvis will be tracked and the overlay will track with the pelvis when the real time images are displayed. If the target is on the pelvis, the pelvis can be moved for a same effect. For example, in real time and in response to a relative movement of the anatomical structure and the optical sensor unit in the real 3D space, wherein a pose of the respective target associated with the anatomical structure continuously indicates a position and orientation of the anatomical structure in the real 3D space, the computing unit may determine a moved position and orientation of the anatomical structure using the images received from the optical sensor unit, update the aligning of the augmented reality overlay relative to the moved position and orientation of the anatomical structure to determine a moved desired position and orientation of the augmented reality overlay; and provide the augmented reality overlay for display in the moved desired position and orientation.

It will be understood that depending on the target configuration employed during a procedure, relative movement of the anatomical structure and optical sensor unit may be restricted. If a target is attached to an anatomical structure whereby movement of the structure moves the target, then the structure may be moved. If the structure is associated in another manner, for example, the target is coupled to a stationary structure such as the OR table and the association is a notional one, premised on the fact that the anatomical structure associated with the target will not be moved during the tracking, then the structure is to remain in its initial position of registration in the real 3D space and the optical sensor unit alone is free to be moved.

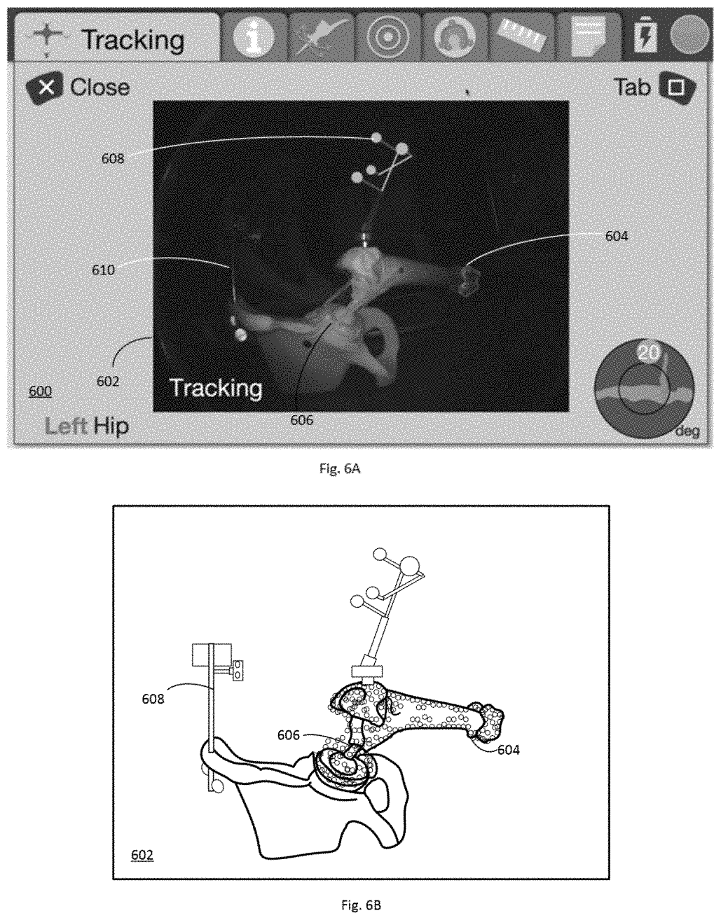

It is understood that other bones may be tracked such as a femur, whether within a THA procedure or a Total Knee Arthroplasty (TKA) procedure. A femur may be registered (not shown) using a femoral target associated with the femur. A femoral overlay may be presented, aligning the 3D model thereof to the desired position associated with the corresponding position of the femur in the computational 3D space. FIG. 6A is a screenshot 600 of a GUI showing a captured video image 602 displayed with an overlay 604 of the pre-operative femur on the femur with replacement implants 606 captured in the video image (in a mock surgery). The overlay 604 of the preoperative femur is defined using stippling (points) through which the anatomy and implants 606 as captured in the real time video image is observed. FIG. 6B is a sketch of video image 602 and overlay 604 of FIG. 6A where the stippling is enlarged for clarity. FIGS. 6A and 6B also show a tracker 608 and a platform 610 on which an optical sensor unit may be mounted.

As noted previously, the overlay may be patient specific, representing patient anatomy that is diseased or not diseased, (e.g. pre-diseased anatomy). Diseased anatomy overlays may be constructed from scans of a patient obtained prior to surgery where the patient exhibits the disease. Pre-diseased anatomy overlays may be constructed from historical scans of the patient before onset of at least some of the disease or from more recent scans that show disease but are edited or otherwise pre-processed, for example, filling in surface, removing or reducing a surface, etc. to define anatomy without disease. In a first example, the anatomy is a knee joint and a disease is degenerative arthritis (essentially worn down cartilage). A knee image ((e.g. computed tomography (CT) or magnetic resonance imaging (MRI) scan) is processed and regions where cartilage is worn down are identified, and virtually filled in by interpolating based on any surrounding healthy tissue. In a second example, the anatomy is a hip joint and the disease is degenerative arthritis, including osteophyte growth (e.g. intra and/or extra acetabular). Pre-osteophyte hip joint geometry is determined based on: surrounding normal bony structures and possibly also from a template of a healthy bone.

The augmented reality overlay may be displayed over the patient's anatomical structure at any time during the surgery. For example, the augmented reality overlay may be displayed prior to treatment of the anatomy (e.g. primary surgical incision, dislocation, removal of a portion of a bone, insertion of an implant or tool), or post-treatment such as over post-treatment anatomy (such as FIGS. 6A-6B, which post-treatment anatomy may include an implant).

In one example, the surgery is a total knee arthroplasty, and the surgical goal is kinematic alignment. The anatomical structure is a femur and the generated overlay is of the distal femur. The overlay may be generated from a overlay model that represents the pre-arthritic knee. The computer implemented method provides a step in which, during femur trialing (i.e. when a provisional implant is fitted to the resected distal femur to confirm fit), the overlay (comprising a pre-arthritic distal femur) is displayed in relation to the provisional implant. A goal of kinematic knee replacement is to exactly replace the bone that is resected, while adjusting for the effects of arthritic disease. The view of the real 3D space comprising a real provisional (or final) implant with an overlay of the pre-arthritic anatomical structure provides a surgeon with information on how well the kinematic alignment goals of the surgery are being achieved, and if the alignment should be adjusted.

When the 3D overlay is a mechanical axis or another axis or plane that is displayed relative to the mechanical axis of the patient, computing unit 106 computes the mechanical axis.

Though not shown, the tracked bone such as a femur may be rotated about a first end thereof (such as rotating within the acetabulum). The rotation may be captured from tracking information received from optical sensor unit 102. A second end location of the femur may be received such as by tracking a probe as it touches points on the end near the knee. Poses of the probe are received and locations in the computational 3D space may be determined. The mechanical axis may be determined by computing unit 106 based on the center of rotation and poses of the probe in the computational 3D space.

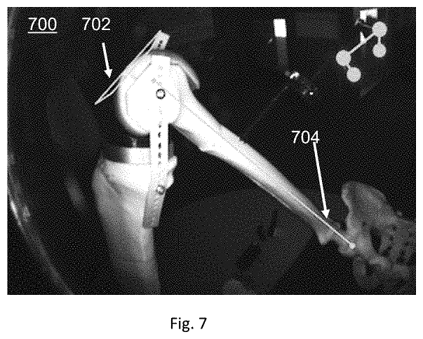

Other planes such as a resection plane may be determined from the mechanical axis. The resection may show angle and depth. Thus the 3D model may be a mechanical axis model and the augmented reality overlay may be an image of a mechanical axis and/or a further axis or plane, a desired location of which is determined relative to a location of the mechanical axis of the anatomical structure. FIG. 7 is a cropped captured video image 700, for display in a GUI such as shown in FIG. 6A, with a cutting plane 702 and mechanical axis 704 showing a hip centre overlayed as guidance in a mock total knee arthroplasty.

An initial location of the resection plane may be determined by computing unit 106 from preset data (example defined to be X mm from the end) or from input received (e.g. via a pull down menu or input form both not shown). The initial location may be moved, for example, in increments or absolutely, in response to input received thereby to adjust the desired position and orientation of the resection plane in the augmented reality overlay. The angle may also be initially defined and adjusted.

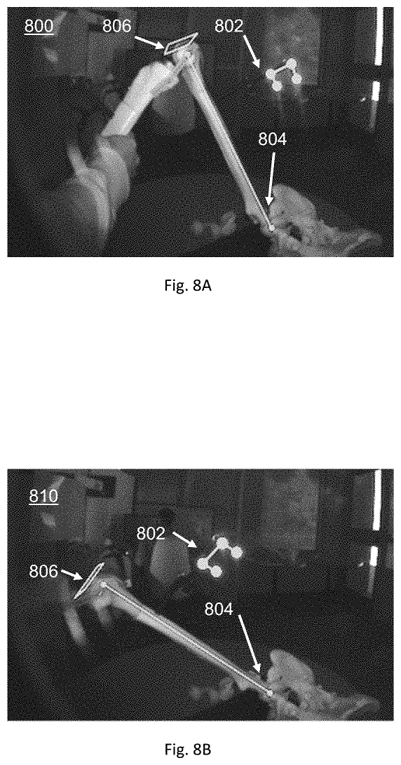

For TKA, for example, a tibia may also be registered (not shown) and a mechanical axis determined for the tibia such as by probing points on the tibia within the knee joint to provide a first end location and providing a second end location by probing points about the ankle end. A tibia overlay may also be rendered and displayed as described in relation to the femur. The overlays may be relative to the mechanical axis and for both bones may be provided in real time, and trackable through knee range of motion. One or both overlays may be shown. The overlays for the femur and tibia for knee applications may show or confirm desired bony cuts (both angle and depth) on distal femur and proximal tibia (femur: varus/valgus, slope, tibia: varus/valgus, slope). FIGS. 8A and 8B are respective captured video images 800 and 810, for display in a GUI such as shown in FIG. 6A, showing a target 802 coupled to knee anatomy (e.g. a femur) as a knee moves from extension to flexion showing a mechanical axis 804 and resection plane 806 over the real time images of the knee. The anatomy in the captured images of FIGS. 6A, 7 and 8A-8B is a physical model for mock surgery.

Though not shown, the visible images of the real 3D space may be displayed in an enlarged manner, for example, zooming in automatically or on input on a region of interest. Zooming may be performed by the computing unit or other processing so that the field of view of the camera does not shrink and the targets leave the field of view. For example, if tracking a knee thru a range of motion, a blown up view of the knee joint would be helpful. This view as displayed need not include the trackers. The augmented reality overlay is then zoomed (rendered) in an enlarged manner accordingly. The zoomed in view could be either 1) locked in to a particular region of the imager, or 2) locked in to a particular region relative to an anatomy (i.e. adaptively follow the knee joint thru a range of motion).

The two overlays (for the femur and tibia for example) may be visually distinct in colour. Relative movement of the femur and tibia with respective overlays presented may illustrate or confirm pre-planning parameters to ensure the relative location is not too proximate and that there is no intersection. The computing unit may determine a location of each overlay and indicate relative location to indicate at least one of proximity and intersection. For example, the proximate area between the two overlays may be highlighted when a relative location (distance) is below a threshold. Highlighting may include a change in colour of the regions of the overlays that fall below the threshold.

In some embodiments, the overlay may be defined during the procedure, for example, by capturing multiple locations identified by a tracked instrument, such as a probe, as it traces over an object. The object may be a portion of a patient' anatomy and the traced portion of the anatomy need not be one that is being tracked while tracing.

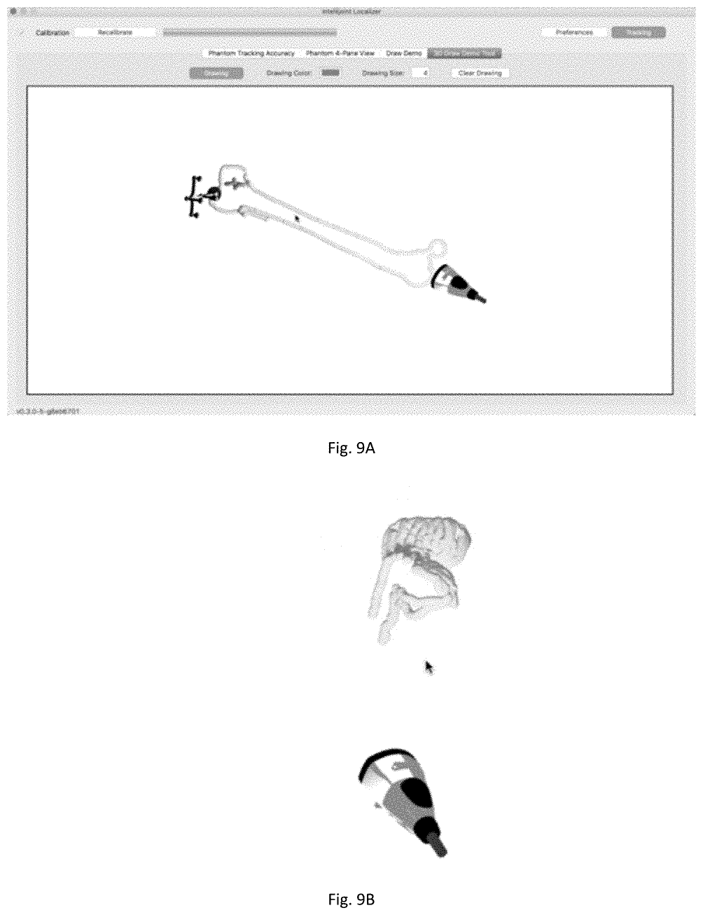

FIGS. 9A and 9B illustrate a capture of a drawing (without the real time images of the sensor's field of view and the associated anatomical structure). Computing unit 106 may be invoked to capture the locations and store the same, defining a 3D model. A button or other input device may be invoked to initiate the capture. In one embodiment, the button/input may be held for the duration of the capture, stop capture when released.

Augmented Reality Assisted Registration

Augmented reality overlay may assist registration of patient anatomy. In one embodiment, an overlay may be projected (displayed over real time images of patient anatomy) on the display screen. A target is coupled to an anatomical structure to be registered in the computational 3D space. The patient's structure may be a femur for example and the overlay may be a femoral overlay. The femur is then moved into alignment with the overlay and the pose of the femur is then locked or associated with the current pose of the overlay in the computational 3D space. Thereafter, the femoral overlay tracks with the relative movement of the femur and optical sensor unit in the real 3D space. By way of example, for THA, the optical sensor unit 102 may be coupled to the pelvis 104 and the pelvis 104 registered to system 100 such as previously described. The optical sensor unit 102 is oriented toward the femur with a target coupled to the femur that is in the field of view of optical sensor unit 102. The overlay is displayed.

System 100 defines an initial or registration pose of the overlay in the computational 3D space. The initial pose may be a default position relative to optical sensor unit or registration axes or may be relative to a location of the target attached to femur. This initial pose of the overlay is maintained and the femur may be moved into alignment with the overlay, then “locked in” such as by system 100 receiving a user input to capture the current pose of the femoral target. If a prior registration was performed but was not sufficiently accurate, for example because the overlay and anatomical structure do not appear to be aligned in the display, a re-registration may be performed using this method, adjusting the current registration by moving the patient anatomy (structure with target) while holding the overlay in a current pose until the anatomy and overlay are aligned in the display. The system may be invoked to hold or decouple the overlay from the tracked anatomical structure, such that the initial pose is the current pose for the overlay in the computational 3D space until the anatomical structure is aligned and the system is invoked to lock in the pose of the anatomical structure as moved to the overlay. Thereafter movement of the anatomical structure relative to the optical sensor unit moves the overlay in the display as described above.

The surgeon sees an overlay of where the “system” thinks the femur axes are vs where the femur axes are visually and brings them into alignment.

The augmented reality overlay could be based on a medical image, or could be composed of lines/planes/axes describing the femur (or other applicable anatomical structure).

A femoral center of rotation calculation may be performed by rotating the femur in the acetabulum or acetabular cup and capturing sufficient poses of the femoral target to determine a location of the center of rotation. This location may then be used as a femur registration landmark.

In another embodiment, while patient anatomy remains stationary in the real 3D space, an overlay associated with an anatomical structure to be registered is displayed over the anatomical structure. The pose of overlay in the computational 3D space is associated with a target in the field of view of the sensor (e.g. a registration axis frame with a target or another instrument with a target, or merely the target itself) such that movement of the target in the real 3D space moves the pose of the overlay. Attachment of the target to another mechanical object (e.g. an instrument like the axis frame or a probe, etc.) may assist with precision positional alignment. Once the overlay is aligned with the anatomical structure, the pose of the anatomical structure is registered in the computational 3D space and the pose of the overlay is associated or locked to the anatomical structure. Locking in may be responsive to user input received to capture the current pose.

The initial position of the overlay in the computational 3D space and hence as displayed may be relative to the current pose of the overlay target in the field of view.

If a registration has previously been performed but determined to be misaligned, (see above with reference to the pelvic overlay description and FIG. 4), the initial position may be the current position of the overlay in the computational 3D space. The pose of the overlay target in the real 3D space is associated with the initial position of the overlay and movement of the overlay target moves the overlay in the computational 3D space and as displayed until it is aligned. Once aligned it may be locked in as described.

Initial registration and registration adjustments under these embodiments (i.e. where the overlay is moved or the structure is moved) are performed in up to 6DOF.

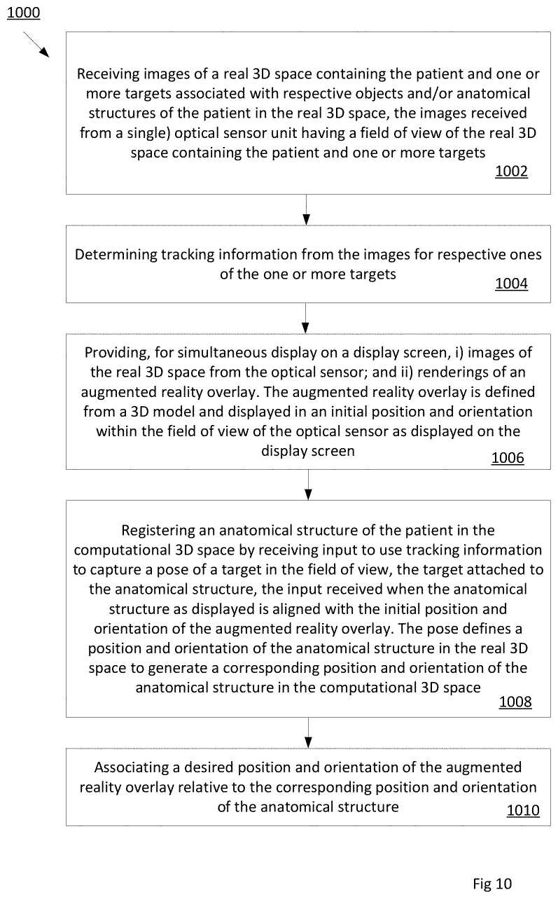

FIG. 10 illustrates a flowchart 1000 of operations to provide augmented reality in relation to a patient in accordance with one embodiment to achieve registration. In this embodiment, an anatomical structure is moved to align with an augmented reality overlay to achieve registration of the anatomical structure to a navigational surgery system. At 1002 at least one processor receives images of a real 3D space containing the patient and one or more targets associated with respective objects and/or anatomical structures of the patient in the real 3D space, the images received from a single optical sensor unit having a field of view of the real 3D space containing the patient and one or more targets. At 1004, tracking information is determined from the images for respective ones of the one or more targets.

At 1006 the computing unit provided, for simultaneous display on a display screen, i) images of the real 3D space from the optical sensor; and ii) renderings of an augmented reality overlay. The augmented reality overlay is defined from a 3D model and displayed in an initial position and orientation within the field of view of the optical sensor unit as displayed on the display screen. At 1008 an anatomical structure of the patient in the computational 3D space is registered by receiving input to use tracking information to capture a pose of a target in the field of view, the target attached to the anatomical structure, the input received when the anatomical structure as displayed is aligned with the initial position and orientation of the augmented reality overlay. The pose defines a position and orientation of the anatomical structure in the real 3D space to generate a corresponding position and orientation of the anatomical structure in the computational 3D space.

At 1010 a desired position and orientation of the augmented reality overlay is associated to the corresponding position and orientation of the anatomical structure.

It is understood that when there is relative movement in the real 3D space, the overlay will move accordingly. For example, in real time and in response to a relative movement of the anatomical structure and the optical sensor unit in the real 3D space, wherein a pose of the anatomical structure target attached to the anatomical structure continuously indicates a position and orientation of the anatomical structure in the real 3D space, the at least one processor will: update the corresponding position and orientation of the anatomical structure by tracking the position and orientation of the anatomical structure in the real 3D space using tracking information; update the desired position and orientation of the augmented reality overlay relative to the corresponding position and orientation of the anatomical structure as updated; and render and provide, for simultaneous display on a display screen, i) images of the real 3D space from the optical sensor; and ii) the augmented reality overlay in response to the desired position and orientation of the augmented reality overlay as updated.

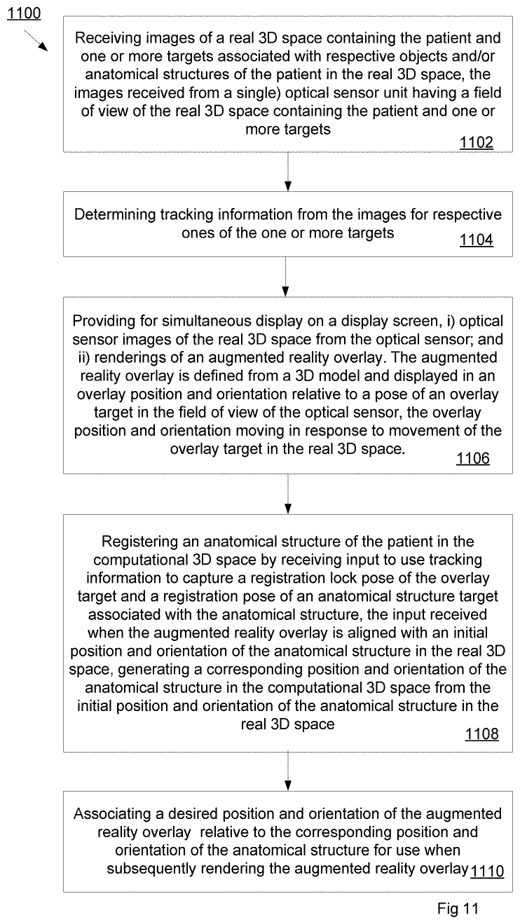

FIG. 11 illustrates a flowchart 1100 for operations to provide augmented reality in relation to a patient to achieve registration. At 1102 at least one processor receives images of a real 3D space containing the patient and one or more targets associated with respective objects and/or anatomical structures of the patient in the real 3D space, the images received from a single optical sensor unit having a field of view of the real 3D space containing the patient and one or more targets. At 1104 tracking information is determined from the images for respective ones of the one or more targets. At 1106, computing unit provides for simultaneous display on a display screen, i) optical sensor images of the real 3D space from the optical sensor; and ii) renderings of an augmented reality overlay. The augmented reality overlay is defined from a 3D model and displayed in an overlay position and orientation relative to a pose of an overlay target in the field of view of the optical sensor, the overlay position and orientation moving in response to movement of the overlay target in the real 3D space.

At 1108 an anatomical structure of the patient is registered in the computational 3D space by receiving input to use tracking information to capture a registration lock pose of the overlay target and a registration pose of an anatomical structure target associated with the anatomical structure, the input received when the augmented reality overlay is aligned with an initial position and orientation of the anatomical structure in the real 3D space, generating a corresponding position and orientation of the anatomical structure in the computational 3D space from the initial position and orientation of the anatomical structure in the real 3D space.

At 1110 in the computational 3D space, a desired position and orientation of the augmented reality overlay is associated relative to the corresponding position and orientation of the anatomical structure for use when subsequently rendering the augmented reality overlay.

Operations may then track and move the overlay as previously described.

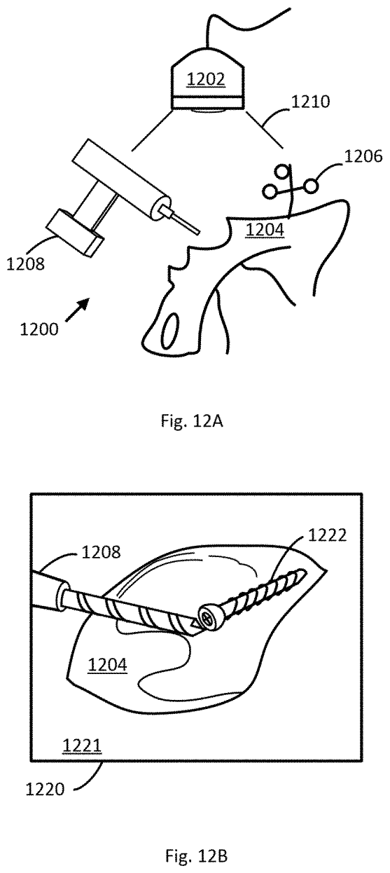

Augmented Reality Overlay for a Planned Position Augmented reality overlays may be employed in many examples. With reference to FIGS. 12A and 12B one further example involves a surgical procedure to place an implant (e.g. an acetabular component or a fixation screw) in a planned position. FIG. 12A shows a sketch of an operating room 1200 including a camera tracking an anatomical structure 1204 via a tracker 1206 and a surgical tool 1208. The surgical tool 1208 is a drill. The overlay may include the planned position of the implant, based on the (prior) registration of the anatomical structure 1204 such as described previously. In one example, a surgical navigation system executing a software workflow may provide a feature for a bone removal step of the procedure to prepare the bone to receive the implant (e.g. acetabular reaming or screw pilot hole drilling). The surgical navigation guidance for this step may comprise displaying (e.g. persistently) the overlay of the planned position of the implant with the real view of the 3D space during bone removal, so as to visually guide the surgeon by visually indicating whether the actual bone removal tool (e.g. reamer or drill) is correctly positioned relative to the planned implant position. FIG. 12B is an illustration of a display screen 1220 showing a video image 1221 of the operating room 1200 including the anatomical structure 1204 from the point of view (and within the field of view 1210) of the camera 1202. Video image 1221 also shows a portion of the surgical tool 1208 as well as the overlay 1222 representing a fixation screw in a planned position. It is understood that the video image 1221 fills the display screen 1220 but may be shown in a portion of the screen. This example of an augmented reality overlay may be advantageous since it does not necessitate tracking a target associated with the surgical tool 1208 to achieve positional guidance.

AR Platform

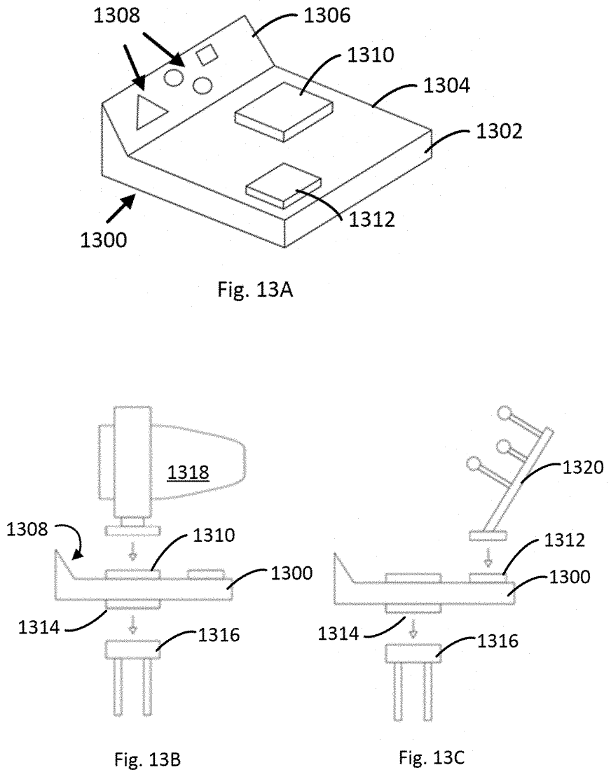

FIG. 13A is a top perspective view of an AR platform 1300 and FIGS. 13B-C are side views of the AR platform 1300 showing how to use the AR platform 1300 to facilitate optical sensor unit attachment to an anatomical structure (not shown in FIGS. 13A-13C) for certain uses during surgery, while allowing the optical sensor unit to be removed (e.g. handheld) for the purposes of augmented reality display. AR platform 1300 comprises a body 1302 with at least one surface (e.g. surfaces 1304 and 1306) having an optically trackable pattern 1308, a repeatable optical sensor mount 1310 and a repeatable target mount 1312. AR Platform 1300 may have a repeatable anatomical structure mount 1314 (e.g. on an underside surface) to mount to a cooperating mount 1316 which may be driven into the anatomical structure or otherwise fixed thereto.

AR platform 1300 is intended to be rigidly mounted to the patient's anatomical structure. The spatial relationship between the optically trackable pattern 1308 and the repeatable target mount 1312 is predefined, and this target-pattern definition is accessible in the memory on the computing unit of the augmented reality navigation system (not shown in FIGS. 13-A-13C). When an optical sensor unit 1318 is mounted to the AR platform 1300 at the repeatable optical sensor mount 1310, the optically trackable pattern 1308 is in the field of view of the optical sensor. The optically trackable pattern 1308 only occupies a portion of the field of view, such that the optical sensor unit 1318 is still able to detect other objects within its field of view (e.g. other targets). The computing unit receives images including the optically trackable pattern features, and performs operations to calculate the pose of the optically trackable pattern. The computing unit performs operations to calculate the pose of the repeatable target mount based on the pose of the optically trackable pattern and the target-pattern definition. FIG. 13C shows a mounting of a target 1320 to repeatable tracker mount 1312, for example to enable the optical sensor unit 1318 to be handheld yet still track the anatomical structure to which the AR platform 1300 and hence target 1320 is attached.

Hence in one mode of operation, the optical sensor unit 1318 may be rigidly attached to the patient's anatomical structure via the AR platform 1300. A computational 3D space may be associated with the optical sensor unit 1318. In the augmented reality mode of operation, the optical sensor unit 1318 may be removed from its repeatable optical sensor mount 1310, and a target 1320 may be mounted on the AR platform 1300 on its repeatable target mount 1312. The computational 3D space association may be passed from the optical sensor unit 1318 to the target 1320 (by the operations executing on the computing unit) via the relative pose of the optical sensor unit 1318 and the target 1320, as well as the calculated relationship of the optical sensor unit 1318 to the repeatable target mount 1312 when the optical sensor unit 1318 is mounted to the AR platform 1300.

As a result, a system may operate in two modes of operation with a single computational 3D space associated with the patient: one in which the optical sensor unit 1318 is mounted to the patient (e.g. for navigational purposes, such as acetabular implant alignment in THA); and another in which the optical sensor unit 1318 is not located on the patient, but a tracker 1230 is mounted on the patient (e.g. for augmented reality purposes).

In addition to anatomical structures being registered to a computational 3D space, tools may also be registered to the computational 3D space, and augmented reality overlays based on the tools may be provided.

The augmented reality navigation system (and any associated method) may provide visual information for display comprising: a) The real 3D space; b) an augmented reality overlay of the anatomical structure (note: there may be different variants of this overlay. For example, current anatomy vs pre-disease anatomy); c) an augmented reality overlay of the tool(s); and an augmented reality overlay of a surgical plan (e.g. planned implant positions). These may be shown in various combinations.

A surgical plan may comprise the planned pose of an implant with respect to an anatomical structure (e.g. the planned pose of an acetabular implant with respect to a patient's pelvis). Alternatively, a surgical plan may comprise a “safe zone”, indicative of spatial regions or angles that are clinically acceptable (for example, the “Lewinnek safe zone” that defines acceptable acetabular implant angles relative to a pelvis, or in another example, regions that are sufficiently far away from critical anatomical structures that could be damaged (e.g. spinal cord).

Since the amount of visual information may be overwhelming to viewer, the computer-implemented method may selectively provide visual information. For example, each of the real 3D space, anatomical structure overlay, tool overlay and plan overlay may comprise layers of the displayed composite image, and may be toggled on or off by the user (e.g. using buttons coupled to the optical sensor, by voice command or via a GUI or other control). In another example, the computer-implemented method may access context information (e.g. what step is being performed in the surgical workflow by detected what step of the software workflow the user is at), and automatically set the layers based on the context information. For example, during a verification step of the surgical workflow, the computer-implemented method may be programmed to display the real 3D space (which includes a real view of an implant), and a surgical plan layer, such that the viewer may visually compare the real view of the implant with its planned position. In this view the anatomical structure and/or tool overlays would be suppressed to avoid providing excessive visual information.

In one example, the context information used to modify the displayed information is the pose of the optical sensor. The pose of the optical sensor unit may be indicative of the desired display for a viewer. The pose of the optical sensor unit may be with respect to a target, or with respect to an inertial frame (such as the direction of gravity, provided that the optical sensor unit is augmented with gravity sensing capabilities).

In one example, an augmented reality overlay of a surgical plan is provided. The computer-implemented method may be communicatively coupled to a surgical planning module. The surgical planning module may facilitate real-time changes to the surgical plan, and the augmented reality overlay of the surgical plan may be updated accordingly. For example, the surgical plan may be the pose of an implant with respect to a bone. During a surgery, there may be reasons to change an initial pose of the implant with respect to the bone to an updated one. In this case, where the augmented reality overlay comprises the pose of the implant with respect to the bone, the overlay would update from the initial pose to the updated one, responsive to the change in plan.

In one example, the optical sensor unit is coupled to (or comprises) a gravity sensing device, and an overlay is provided for display representing the direction of gravity.

The scope of the claims should not be limited by the embodiments set forth in the examples, but should be given the broadest interpretation consistent with the description as a whole.

What is claimed is:

1. A computer-implemented method to provide augmented reality in relation to a patient, the method comprising:
receiving, by at least one processor, images of a real 3D space containing the patient and one or more targets associated with respective objects and/or anatomical structures of the patient in the real 3D space, the images received from a single optical sensor unit having a field of view of the real 3D space containing the patient and one or more targets;
determining tracking information from the images for respective ones of the one or more targets;
registering an anatomical structure of the patient in a computational 3D space maintained by the at least one processor using tracking information for a respective target associated with the anatomical structure, generating a corresponding position and orientation of the anatomical structure in the computational 3D space from a position and orientation of the anatomical structure in the real 3D space;
aligning an overlay model of an augmented reality overlay to a desired position and orientation in the computational 3D space relative to the corresponding position and orientation of the anatomical structure; and
rendering and providing the augmented reality overlay for display on a display screen in the desired position and orientation.

2. The method of claim 1 comprising providing the images of the real 3D space for display on the display screen to simultaneously visualize the anatomical structure and the augmented reality overlay.

3. The method of claim 1, wherein the optical sensor unit comprises calibration data to determine 3D measurements from the images of the real 3D space provided by the optical sensor unit in 2D and the step of determining tracking information comprises using by the at least one processor the calibration data to determine the tracking information.

4. The method of claim 1, comprising, in real time and in response to a relative movement of the anatomical structure and the optical sensor unit in the real 3D space, wherein a pose of the respective target associated with the anatomical structure continuously indicates a position and orientation of the anatomical structure in the real 3D space:
determining a moved position and orientation of the anatomical structure in the real 3D space using the images received from the optical sensor unit;
updating the aligning of the augmented reality overlay relative to the moved position and orientation of the anatomical structure to determine a moved desired position and orientation of the augmented reality overlay; and
providing the augmented reality overlay for display in the moved desired position and orientation.

5. The method of claim 4 wherein the respective target associated with the anatomical structure is either 1) attached to the anatomical structure such that one or both of the optical sensor unit and anatomical structure are free to move in the real 3D space or 2) attached to another object while the location of anatomical structure remains constant in the real 3D space and the optical sensor unit alone is free to move in the real 3D space.

6.-10. (canceled)

11. The method of claim 1, wherein the overlay model is a 3D model of a mechanical axis model and the augmented reality overlay is an image of a mechanical axis and/or a further axis or plane, a location of which is determined relative to a location of the mechanical axis of the anatomical structure.

12. The method of claim 11, comprising determining the mechanical axis of the anatomical structure using tracking information obtained from target images as the anatomical structure is rotated about an end of the anatomical structure.

13. The method of claim 12, wherein the further axis and/or plane is a resection plane.

14. The method of claim 13, wherein the location of the resection plane along the mechanical axis model is adjustable in response to user input thereby to adjust the desired position and orientation of the resection plane in the augmented reality overlay.

15. The method of claim 11, wherein the bone is a femur.

16. The method of claim 15, comprising:
registering a tibia of a same leg of the patient in the computational 3D space, the tibia coupled to a tibia target of the one or more targets, the at least one processor determining a position and orientation of the tibia in the real 3D space to generate a corresponding position and orientation of the tibia in the computational 3D space from tracking information determined from images of the tibia target;
aligning a second overlay model of a second augmented reality overlay to a second desired position and orientation in the computational 3D space relative to the corresponding position and orientation of the tibia;
providing the second augmented reality overlay for display on the display screen in the second desired position and orientation.

17. The method of claim 16, wherein registering uses images of one of the targets attached to a probe where the probe identifies first representative locations on the tibia with which to define a first end of the tibia and second identifying locations about an ankle of the patient with which to define a second end and a mechanical axis of the tibia.

18. The method of claim 16, comprising:
tracking movement of the position and orientation of the tibia in the real 3D space;
updating the corresponding position and orientation of the tibia in response to the movement of the position and orientation of the tibia in the real 3D space;
updating the aligning of the second augmented reality overlay relative to the position and orientation of the tibia as moved to determine the second desired position and orientation as moved; and
providing the second augmented reality overlay for display in the second desired position and orientation as moved.

19. The method of claim 18, comprising determining a location of each of the augmented reality overlay of the femur and the augmented reality overlay of the tibia and indicating a relative location to one another to denote at least one of proximity and intersection.

20. (canceled)

21. The method of claim 1, wherein the anatomical structure is surgically modified and wherein the overlay model is a 3D model of a generic or patient-specific human anatomical structure prior to replacement by a prosthetic implant and the augmented reality overlay is an image representing a generic or a patient-specific human anatomical structure respectively; and wherein the method comprises providing images of the patient for display on the display screen to simultaneously visualize the anatomical structure and the augmented reality overlay.

22. The method of claim 1, wherein the overlay model is a 3D model defined from pre-operative images of the patient.

23. The method of claim 1, wherein the overlay model is a 3D model defined from pre-operative images of the patient and the pre-operative images of the patient show a diseased human anatomical structure and wherein the overlay model represents the diseased human anatomical structure without a disease.

24. A computer-implemented method to provide augmented reality in relation to a patient, the method comprising:
receiving, by at least one processor, images of a real 3D space containing the patient and one or more targets associated with respective objects and/or anatomical structures of the patient in the real 3D space, the images received from a single optical sensor unit having a field of view of the real 3D space containing the patient and one or more targets;
determining tracking information from the images for respective ones of the one or more targets;
providing, for simultaneous display on a display screen, i) images of the real 3D space from the optical sensor; and ii) renderings of an augmented reality overlay; wherein the augmented reality overlay is defined from an overlay model in a computational 3D space and displayed in an initial position and orientation within the field of view of the optical sensor unit as displayed on the display screen;
registering, by the at least one processor, an anatomical structure of the patient in the computational 3D space by receiving input to use tracking information to capture a pose of one of the targets in the field of view, the one of the targets attached to the anatomical structure, the input received when the anatomical structure as displayed is aligned with the initial position and orientation of the augmented reality overlay; and wherein the pose defines a position and orientation of the anatomical structure in the real 3D space to generate a corresponding position and orientation of the anatomical structure in the computational 3D space; and
associating, in the computational 3D space, a desired position and orientation of the augmented reality overlay relative to the corresponding position and orientation of the anatomical structure.

25. A computer-implemented method to provide augmented reality in relation to a patient, the method comprising:
receiving, by at least one processor, images of a real 3D space containing the patient and one or more targets associated with respective objects and/or anatomical structures of the patient in the real 3D space, the images received from a single optical sensor unit having a field of view of the real 3D space containing the patient and one or more targets;
determining tracking information from the images for respective ones of the one or more targets;
providing, for simultaneous display on a display screen, i) optical sensor images of the real 3D space from the optical sensor unit; and ii) renderings of an augmented reality overlay; wherein the augmented reality overlay is defined from an overlay model in a computational 3D space and displayed in an overlay position and orientation relative to a pose of an overlay target in the field of view of the optical sensor unit, the overlay position and orientation moving in response to movement of the overlay target in the real 3D space;
registering, by the at least one processor, an anatomical structure of the patient in the computational 3D space by receiving input to use tracking information to capture a registration lock pose of the overlay target and a registration pose of an anatomical structure target associated with the anatomical structure, the input received to affect an aligning when said augmented reality overlay is aligned with an initial position and orientation of the anatomical structure in the real 3D space, generating a corresponding position and orientation of the anatomical structure in the computational 3D space comprising the aligning from the initial position and orientation of the anatomical structure in the real 3D space; and
associating, in the computational 3D space, a desired position and orientation of the augmented reality overlay relative to the corresponding position and orientation of the anatomical structure for use when subsequently rendering the augmented reality overlay.

26. The method of claim 24, comprising, in real time and in response to a relative movement of the anatomical structure and the optical sensor unit in the real 3D space, wherein a pose of the anatomical structure target associated with the anatomical structure continuously indicates a position and orientation of the anatomical structure in the real 3D space:
determining a moved position and orientation of the anatomical structure using the images received from the optical sensor unit;
updating the aligning of the augmented reality overlay relative to the moved position and orientation of the anatomical structure to determine a moved desired position and orientation of the augmented reality overlay; and
rendering and providing, for simultaneous display on the display screen, i) images of the real 3D space from the optical sensor unit; and ii) the augmented reality overlay in response to the moved desired position and orientation of the augmented reality overlay.

27. The method of claim 24 comprising performing an initial registration of the anatomical structure, an initial aligning of the augmented reality overlay to the anatomical structure and an initial rendering and providing such that the augmented reality overlay and anatomical structure are misaligned in the images of the real 3D space when displayed.

28. (canceled)

29. (canceled)

30. A navigational surgery system comprising a computing unit, an optical sensor unit and one or more targets for tracking objects by the optical sensor unit providing tracking images having tracking information for said targets and visible images of a procedure in a field of view of the optical sensor unit to the computing unit, the computing unit having at least one processor configured to:
receive by the at least one processor images of a real 3D space containing the patient and the one or more targets associated with respective objects and/or anatomical structures of the patient in the real 3D space, the images received from a single optical sensor of the optical sensor unit having a field of view of the real 3D space;
determine tracking information from the images for respective ones of the one or more targets;
provide, for simultaneous display on a display screen, i) images of the real 3D space from the single optical sensor; and ii) renderings of an augmented reality overlay; wherein the augmented reality overlay is defined from an overlay model in a computational 3D space and displayed in an initial position and orientation within the field of view of the optical sensor unit as displayed on the display screen;
register by the at least one processor an anatomical structure of the patient in the computational 3D space by receiving input to use tracking information to capture a pose of one of the targets in the field of view, the one of the targets attached to the anatomical structure, the input received when the anatomical structure as displayed is aligned with the initial position and orientation of the augmented reality overlay; and wherein the pose defines a position and orientation of the anatomical structure in the real 3D space to generate a corresponding position and orientation of the anatomical structure in the computational 3D space; and
associate in the computational 3D space a desired position and orientation of the augmented reality overlay relative to the corresponding position and orientation of the anatomical structure.

31. The navigational surgery system of claim 30 comprising:
a platform to selectively, removably and rigidly attach one of the optical sensor unit and one of the trackers to an anatomical structure of the anatomy of the patient, the platform comprising a body having at least one surface, the at least one surface configured to provide an optically trackable pattern, a repeatable optical sensor mount and a repeatable target mount, wherein the optically trackable pattern extends into a field of view of the optical sensor unit when mounted to the platform; and wherein:
a spatial relationship between the optically trackable pattern and the repeatable target mount is predefined by a target-pattern definition; and
the computing unit is configured to:
receive first images including the optically trackable pattern features when the optical sensor unit is mounted to the platform;
perform operations to calculate a pose of the optically trackable pattern;
perform operations to calculate the pose of the repeatable target mount based on the pose of the optically trackable pattern and the target-pattern definition;
receive second images when the optical sensor unit is removed from the platform and one of the trackers is mounted to the platform, the second images including the one of the trackers mounted to the platform; and
track the anatomical structure to which the one of the trackers is attached.
